(19) United States
(12) Patent Application Publication
(10) Pub. No.: US 2006/0259061 A1
(43) Pub. Date: Nov. 16, 2006

(54) Expandable sheath for percutaneous upper gastrointestinal tract access

(76) Inventors: George F. Kick, Casa Grande, AZ (US); Jay Lenker, Laguna Beach, CA (US); Edward J. Nance, Corona, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/409,723

(22) Filed: Apr. 24, 2006

Related U.S. Application Data
(60) Provisional application No. 60/674,228, filed on Apr. 22, 2005.

(51) Int. Cl. A61M 29/00

(52) U.S. Cl. 606/191

(57) ABSTRACT

Disclosed is an expandable percutaneous sheath, for introduction into the body while in a first, low cross-sectional area configuration, and subsequent expansion of at least a part of the distal end of the sheath to a second, enlarged cross-sectional configuration. The sheath is configured for use in the upper gastrointestinal tract and has utility in the performance of procedures in the esophagus and stomach. The access route is through the anterior abdominal wall to the stomach. The distal end of the sheath is maintained in the first, low cross-sectional configuration during advancement through the abdominal wall and into the stomach. The distal end of the sheath is subsequently expanded using a radial dilatation device. In an exemplary application, the sheath is utilized to provide access for a diagnostic or therapeutic procedure such as diagnosis and repair of gastro esophageal reflux disease. The sheath further can be secured within the gastrointestinal system and be used to draw the stomach wall against the abdominal wall.

### PRIORITY CLAIM

This application claims the priority benefit under 35 U.S.C. § 119(e) of Provisional Application 60/674,228 filed Apr. 22, 2005.

### BACKGROUND OF THE INVENTION

1. Field of the Invention

The invention relates to medical devices and methods and, more particularly, to devices and methods for accessing the esophagus and stomach.

2. Description of the Related Art

The lower esophageal sphincter (LES) is a ring of increased thickness in the circular, smooth muscle layer of the esophagus. At rest, the lower esophageal sphincter maintains a high-pressure zone between 15 and 30 mm Hg above intragastric pressures. The lower esophageal sphincter relaxes before the esophagus contracts, and allows food to pass through to the stomach. After food passes into the stomach, the sphincter constricts to prevent the contents from regurgitating into the esophagus. The resting tone of the LES is maintained by myogenic (muscular) and neurogenic (nerve) mechanisms. The release of acetylcholine by nerves maintains or increases lower esophageal sphincter tone. It is also affected by different reflex mechanisms, physiological alterations, and ingested substances. The release of nitric oxide by nerves relaxes the lower esophageal sphincter in response to swallowing, although transient lower esophageal sphincter relaxations may also manifest independently of swallowing. This relaxation is often associated with transient gastro esophageal reflux in normal people.

Gastro esophageal reflux disease, commonly known as GERD, results from incompetence of the lower esophageal sphincter, located just above the stomach in the lower part of the esophagus. Acidic stomach fluids may flow retrograde across the incompetent lower esophageal sphincter into the esophagus. The esophagus, unlike the stomach, is not capable of handling highly acidic contents so the condition results in the symptoms of heartburn, chest pain, cough, difficulty swallowing, or regurgitation. These episodes can ultimately lead to injury of the esophagus. GERD affects a large proportion of the population and mild cases can be treated with lifestyle modifications and pharmaceutical therapy. Patients that are resistant to pharmaceutical therapy or lifestyle changes are candidates for surgical repair of the lower esophageal sphincter. The most common surgical repair, called fundoplication surgery, generally involves manipulating the diaphragm, wrapping the upper portion of the stomach, the fundus, around the lower esophageal sphincter, thus tightening the sphincter, and reducing the circumference of the sphincter so as to eliminate the incompetence. The hiatus, or opening in the diaphragm is reduced in size and secured with 2 to 3 sutures to prevent the fundoplication from migrating into the chest cavity. The repair can be attempted through open surgery, laparoscopic surgery, or an endoscopic, or endoluminal, approach by way of the throat and the esophagus. The open surgical repair procedure, most commonly a Nissen fundoplication, is effective but entails a substantial insult to the abdominal tissues, a risk of anesthesia-related iatrogenic injury, a 7 to 10 day hospital stay, and a 6 to 12 week recovery time, at home. The open surgical procedure is performed through a large incision in the middle of the abdomen, extending from just below the ribs to the umbilicus (belly button).

Very recently, endoscopic techniques for the treatment of GERD have been developed. Laparoscopic repair of GERD has the promise of a high success rate, currently 90% or greater, and a relatively short recovery period due to minimal tissue trauma. Laparoscopic Nissen fundoplication procedures have reduced the hospital stay to an average of 3 days with a 3 week recovery period at home. Another type of laparoscopic procedure involves the application of radio-frequency waves to the lower part of the esophagus just above the sphincter. The waves cause damage to the tissue beneath the esophageal lining and a scar (fibrosis) forms. The scar shrinks and pulling on the surrounding tissue, thereby tightening the sphincter and the area above it. A third type of endoscopic treatment involves the injection of material into the esophageal wall in the area of the lower esophageal sphincter. This increases the pressure in the lower esophageal sphincter and prevents reflux.

One laparoscopic technique that appears to show promise for GERD therapy involves approaching the esophageal sphincter from the downstream, or stomach, side and performing a circumference reducing tightening of the sphincter. However current access devices are inadequate for enabling placement of instruments within the stomach through a percutaneous puncture. Current laparoscopic access devices allow for 10-mm to 15-mm inside diameter sheaths to be introduced into the abdomen. However, these sheaths have no provision to be introduced into the stomach and seal the wall of the stomach against the loss of acidic contents. Furthermore, current sheaths may dilate the stomach wall but cause trauma, which may not properly heal upon sheath removal, given the acidic nature of the contents.

Further reading related to the pathophysiology of GERD includes Mechanisms of Gastro-esophageal Reflux in Patients with Reflux Esophagitis, New England Journal of Medicine 1982;307:1547-1552, Dodds W. J.; Dent J.; Hogan W. J.; Helm J. F.; Hauser R.; Patel G. K.; Egide M. S, The Physiology and Patho-physiology of Gastric-emptyinq in Humans, Gastroenterology 1984;86:1592-1610, and Minami H.; Mccallum R. W., Gastro-esophageal Reflux-Pathogenesis, Diagnosis, and Therapy, Annals of Internal Medicine 1982;97:93-103, Richter J. E.; Castell D. O, the entirety of these articles of which are hereby incorporated by reference herein.

Evidence indicates that up to 36% of otherwise healthy Americans suffer from heartburn at least once a month, and that 7% experience heartburn as often as once a day. It has been estimated that approximately 2% of the adult population suffers from GERD, based on objective measures such as endoscopic or histological examinations. The incidence of GERD increases markedly after the age of 40, and it is not uncommon for patients experiencing symptoms to wait years before seeking medical treatment.

### SUMMARY OF THE INVENTION

A need, therefore, remains for improved access technology, which allows a device to be percutaneously or surgically introduced, advanced into the stomach, and oriented to perform repair of the esophageal sphincter. The device would advantageously further permit dilation of the stomach wall so that the sheath could pass relatively large diameter instruments or catheters. Such large dilations of the tissues of the stomach wall would advantageously be performed in such a way that the residual defect is minimized when the device is removed. In one preferred embodiment, the catheter or sheath would be able to enter a vessel or body lumen with a diameter of 3 to 12 French or smaller, and be able to pass instruments through a central lumen that is 15 to 30 French. The sheath or catheter would be capable of gently dilating the stomach wall and of permitting the exchange of instrumentation therethrough without being removed from the body. The sheath or catheter would also be maximally visible under fluoroscopy and would be relatively inexpensive to manufacture. The sheath or catheter would be kink resistant, provide a stable or stiff platform for esophageal sphincter repair, and minimize abrasion and damage to instrumentation being passed therethrough. The sheath or catheter would further minimize the potential for injury to body lumen or cavity walls or surrounding structures. The sheath or catheter would further possess certain steering capabilities so that it could be deflected to face the esophageal sphincter once placed within the stomach. Finally, the sheath or catheter would possess the ability to seal the stomach penetration and hold the stomach wall against the anterior wall of the abdomen.

One aspect of the present invention comprises an expandable endogastric access sheath for providing minimally invasive access to the upper digestive tract, the sheath. The sheath includes an axially elongate sheath with a proximal end, a distal end, and a central lumen that extends therethrough. A distal region of the sheath being is located generally at the distal end of the sheath and is configured to be is expandable in circumference in response to outward pressure applied therein. A hub is coupled to the proximal end of the sheath tube. An obturator is positioned in the central lumen. The obturator is configured to occlude the central lumen of the sheath during insertion. The obturator includes a hub, a guidewire lumen extending through the obturator, and an expandable portion configured to expand the distal region of the sheath from a collapsed configuration to an expanded configuration. A radially expandable distal anchor is positioned on the sheath and a radially expandable proximal anchor positioned on the sheath proximal to the distal anchor.

Another aspect of the present invention is a method of proving access to a stomach of a patient. The method comprises inserting a guidewire through the abdominal wall into the stomach and inserting a sheath with at least a distal region in a collapsed configuration and with a pre-inserted dilator positioned within a lumen in the sheath into the patient over the guidewire. The sheath is advanced to a treatment or diagnostic site within the stomach or upper digestive tract. The distal region of the sheath is expanded with the dilator. The dilator is collapses and the removed from the sheath. An instrument is inserted through the lumen of the sheath into the stomach. A therapy or diagnosis is performed with the instrument. The sheath is removed from the patient.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a front view schematic representation of the human upper digestive system including the esophagus and the stomach;

FIG. 2 is a front view schematic representation of the human upper digestive system with acid reflux occurring through an incompetent lower esophageal sphincter;

FIG. 3 is a front view schematic representation of the human upper digestive system with an expandable sheath advanced into the stomach, according to an embodiment of the invention;

FIG. 4 is an illustration of the stomach, looking from anterior to posterior, with the expandable sheath articulated and positioned within the stomach, oriented toward the lower esophageal sphincter, and the guidewire removed, according to an embodiment of the invention;

FIG. 5 is an illustration, looking from anterior to posterior, of the stomach with the expandable sheath positioned at the stomach and the internal fixation device expanded, according to an embodiment of the invention;

FIG. 6 is a lateral cross-sectional illustration of the stomach and abdominal wall with the internal anchor expanded, the external anchor expanded and moved distally to clamp the stomach wall to the abdominal wall, according to an embodiment of the invention;

FIG. 7 is an illustration of the stomach and abdomen, looking from anterior to posterior, with the expandable sheath dilated at its distal end by the dilator, according to an embodiment of the invention;

FIG. 8 is an illustration of the stomach with the expandable dilator withdrawn from the sheath leaving a large central lumen for instrument passage into the stomach and esophagus, according to an embodiment of the invention;

FIG. 9 is an illustration of the stomach, looking from anterior to posterior, with a therapeutic catheter advanced through the central lumen of the expanded sheath into the lower esophageal sphincter, according to an embodiment of the invention;

FIG. 10 is a cross-sectional illustration of the expandable sheath showing a deflection mechanism, according to an embodiment of the invention;

FIG. 11 is a cut away illustration of the expandable sheath showing the internal and external anchor mechanisms, according to an embodiment of the invention;

FIG. 12A illustrates a side view of a collapsed, non-expanded gastric sheath, according to an embodiment of the invention;

FIG. 12B illustrates a side view of an expanded gastric sheath, according to an embodiment of the invention;

FIG. 12C illustrates a side view of an expanded gastric sheath with the dilator removed, according to an embodiment of the invention;

FIG. 13 illustrates a side view of a dilator for an expandable gastric sheath, according to an embodiment of the invention;

FIG. 14A illustrates a lateral cross-section of the proximal region of the expandable gastric sheath, according to an embodiment of the invention; and

FIG. 14B illustrates a lateral cross-section of the distal region of the expandable gastric sheath in its non-expanded configuration, according to an embodiment of the invention.

### DETAILED DESCRIPTION OF THE PREFERRED EMBODIMENTS

The invention may be embodied in other specific forms without departing from its spirit or essential characteristics. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

In the description below, the term catheter or a sheath will be used to describe an axially elongate hollow tubular structure having a proximal end and a distal end. In many embodiments, the axially elongate structure further has a longitudinal axis and has an internal through lumen that extends from the proximal end to the distal end for the passage of instruments, fluids, tissue, or other materials. The axially elongate hollow tubular structure can be generally flexible and capable of bending, to a greater or lesser degree, through one or more arcs in one or more directions perpendicular to the main longitudinal axis. As is commonly used in the art of medical devices, the proximal end of the device is that end that is closest to the user, typically a surgeon, or gastroenterologist. The distal end of the device is that end closest to the patient or that is first inserted into the patient. A direction being described as being proximal to a certain landmark will be closer to the user, along the longitudinal axis, and further from the patient than the specified landmark. The diameter of a catheter is often measured in “French Size” which can be defined as 3 times the diameter in millimeters (mm). For example, a 15 French catheter is 5 mm in diameter. The French size is designed to approximate the circumference of the catheter in mm and is often useful for catheters that have non-circular cross-sectional configurations. While the original measurement of “French” used π (3.14159. . . ) as the conversion factor between diameters in millimeters (mm) and French, the system has evolved today to where the conversion factor is 3.0.

As sill be explained below, in one embodiment, a radially expanding access sheath is used to provide access to the stomach by way of a laparoscopic puncture, advancement through the stomach wall, and final placement and orientation in the stomach lumen. In an embodiment, the sheath can have an introduction outside diameter that ranged from 3 to 12 French with a preferred range of 5 to 10 French. The diameter of the sheath can be expandable to permit instruments ranging up to 30 French to pass therethrough, with a preferred range of between 3 and 20 French. The sheath can have a working length ranging between 10-cm and 50-cm with a preferred length of 12-cm to 25-cm. The ability to pass larger, more innovative, instruments through a catheter introduced with a small outside diameter is derived from the ability to atraumatically, radially expand the distal end of the catheter or sheath to create a larger through lumen to access the stomach. The expandable distal end of the sheath can comprise between 5% and 95% of the overall working length of the catheter. The proximal end of the catheter is generally larger than the distal end to provide for pushability, torqueaqbility, steerability, control, and the ability to pass large diameter instruments therethrough. In an embodiment, the sheath can be routed to its destination over one or more already placed guidewires with a diameter ranging up to 0.040 inches and generally approximating 0.038 inches in diameter.

One embodiment percutaneous access system for providing minimally invasive access to structures within the upper gastrointestinal system includes an access sheath comprising an axially elongate tubular body that defines a lumen extending from the proximal end to the distal end of the sheath. At least a portion of the distal end of the elongate tubular body is expandable from a first, smaller cross-sectional profile to a second, greater cross-sectional profile. In one embodiment, the first, smaller cross-sectional profile is created by making longitudinally axial oriented folds in the sheath material. These folds can be located in only one circumferential position on the sheath, or there may be a plurality of such folds or longitudinally oriented crimps in the sheath. The folds reduce the circumference of the sheath in its compressed configuration. The folds or crimps may be made permanent or semi-permanent by heat-setting the structure, once folded. In an embodiment, a releasable or expandable jacket is carried by the access sheath to restrain at least a portion of the elongate tubular structure in the first, smaller cross-sectional profile. In another embodiment, the jacket is removed prior to inserting the sheath into the patient. In an embodiment, the elongate tubular body is sufficiently pliable to allow the passage of objects or instruments having a maximum cross-sectional size larger than an inner diameter of the elongate tubular body in the second, greater cross-sectional profile. The adaptability to objects of larger dimension is accomplished by pliability or re-shaping of the cross-section to the larger dimension in one direction accompanied by a reduction in dimension in a lateral direction. The adaptability may also be generated through the use of malleable or elastomerically deformable sheath material. The malleable sheath material can be derived from a single tube such as polytetrafluoroethylene (PTFE) or it can be derived from a malleable reinforcement, typically annealed metal, encapsulated within a plastically or elastomerically deformable polymer casing.

In another embodiment of the invention, a transluminal access sheath assembly for providing minimally invasive access comprises an elongate tubular member having a proximal end and a distal end and defining a working inner lumen. In such an embodiment, the tubular member can comprise a folded or creased sheath that can be expanded by a dilatation balloon. The dilatation balloon, if filled with fluids, preferably liquids and further preferably radiopaque liquids, at appropriate pressure, can generate the force to radially dilate or expand the sheath. The dilatation balloon and delivery catheter is removable to permit subsequent instrument passage through the sheath. Longitudinal runners can be disposed within the sheath to serve as tracks for instrumentation, which further minimize friction while minimizing the risk of catching the instrument on the expandable plastic tubular member. Such longitudinal runners are preferably circumferentially affixed within the sheath so as not to shift out of alignment. In yet another embodiment, the longitudinal runners may be replaced by longitudinally oriented ridges (thick areas) and valleys (thin areas), termed flutes, which are integral to the wall of the sheath tubing. The flutes, or runners, can be oriented along the longitudinal axis of the sheath, or they can be oriented in a spiral, or rifled, fashion.

In many embodiments, the proximal end of the access assembly, apparatus, or device is preferably fabricated as a structure that is flexible, resistant to kinking, and further retains both column strength and torqueability. Such structures include tubes fabricated with coils or braided reinforcements and preferably comprise inner walls that prevent the reinforcing structures from protruding, poking through, or becoming exposed to the inner lumen of the access apparatus. In another embodiment, the outer wall layer is sufficiently strong as to prevent reinforcing material from protruding through beyond the outer surface of the outer layer and into tissue, which could be damaged from such protrusions. Such proximal end configurations may be single lumen, or multi-lumen designs, with a main lumen suitable for instrument, guidewire, endoscope, or obturator passage and additional lumens being suitable for control and operational functions such as balloon inflation. Such proximal tube assemblies can be affixed to the proximal end of the distal expandable segments described heretofore. In an embodiment, the proximal end of the catheter includes an inner layer of thin polymeric material, an outer layer of polymeric material, and a central region comprising a coil, braid, stent, plurality of hoops, or other reinforcement. It is beneficial to create a bond between the outer and inner layers at a plurality of points, most preferably at the interstices or perforations in the reinforcement structure, which is generally fenestrated. Such bonding between the inner and outer layers causes a braided structure to lock in place. In another embodiment, the inner and outer layers are not fused or bonded together in at least some, or all, places. When similar materials are used for the inner and outer layers, the sheath structure can advantageously be fabricated by fusing of the inner and outer layer to create a uniform, non-layered structure surrounding the reinforcement. The polymeric materials used for the outer wall of the jacket are preferably elastomeric to maximize flexibility of the catheter or sheath. The polymeric materials used in the composite catheter inner wall may be the same materials as those used for the outer wall, or they may be different. In another embodiment, a composite tubular structure can be co-extruded by extruding a polymeric compound with a stent, braid, or coil structure embedded therein. The reinforcing structure is preferably fabricated from annealed metals, such as fully annealed stainless steel, titanium, or the like. In this embodiment, once expanded, the folds or crimps can be held open by the reinforcement structure embedded within the sheath, wherein the reinforcement structure is malleable but retains sufficient force to overcome any forces imparted by the sheath tubing.

In certain embodiments, it is beneficial that the sheath comprise a radiopaque marker or markers. The radiopaque markers may be affixed to the non-expandable portion or they may be affixed to the expandable portion. Markers affixed to the radially expandable portion preferably do not restrain the sheath or catheter from radial expansion or collapse. Markers affixed to the non-expandable portion, such as the catheter shaft of a balloon dilator may be simple rings that are not radially expandable. Radiopaque markers include shapes fabricated from malleable material such as gold, platinum, tantalum, platinum iridium, and the like. Radiopacity can also be increased by vapor deposition coating or plating metal parts of the catheter with metals or alloys of gold, platinum, tantalum, platinum-iridium, and the like. Expandable markers, suitable for the radially expandable distal region, may be fabricated as undulated or wavy rings, bendable wire wound circumferentially around the sheath. The expandable markers can also be structures such as are found commonly on stents, grafts, stent-grafts, or catheters used for endovascular access in the body. Expandable radiopaque structures may also include disconnected or incomplete surround shapes affixed to the surface of a sleeve or other expandable shape. Non-expandable structures include circular rings or other structures that completely surround the catheter circumferentially and are strong enough to resist expansion. In another embodiment, the polymeric materials of the catheter or sheath may be loaded with radiopaque filler materials such as, but not limited to, bismuth salts, barium salts, or the like, at percentages ranging from 1% to 50% by weight in order to increase radiopacity. The radiopaque markers allow the sheath to be guided and monitored using fluoroscopy. The sheath is configured to accept flexible endoscopic equipment to permit visualization of any procedures performed at or near the distal end of the sheath.

In order to provide radial or circumferential expansive translation of the reinforcement, it may be beneficial not to completely bond the inner and outer layers together, thus allowing for some motion of the reinforcement in translation as well as the normal circumferential expansion. Regions of non-bonding may be created by selective bonding between the two layers or by creating non-bonding regions using a slip layer fabricated from polymers, ceramics or metals. Radial expansion capabilities are important because the proximal end needs to transition to the distal expansive end and, to minimize manufacturing costs, the same catheter may be employed at both the proximal and distal end, with the expansive distal end undergoing secondary operations to permit radial or diametric expansion.

In another embodiment, the distal end of the catheter is fabricated using an inner tubular layer, which is thin and lubricious. This inner layer is fabricated from materials such as, but not limited to, FEP, PTFE, polyamide, polyethylene, polypropylene, Pebax, Hytrel, and the like. The reinforcement layer comprises a coil, braid, stent, or plurality of expandable, foldable, or collapsible rings, which are generally malleable and maintain their shape once deformed. Preferred materials for fabricating the reinforcement layer include but are not limited to, stainless steel, tantalum, gold, platinum, platinum-iridium, titanium, nitinol, and the like. The materials are preferably fully annealed or, in the case of nitinol, fully martensitic. The outer layer is fabricated from materials such as, but not limited to, FEP, PTFE, polyamide, polyethylene, polypropylene, polyurethane, Pebax, Hytrel, and the like. The inner layer is fused or bonded to the outer layer through holes in the reinforcement layer to create a composite unitary structure. The structure is crimped radially inward to a reduced cross-sectional area. A balloon dilator is inserted into the structure before crimping or after an initial crimping and before a final sheath crimping. The balloon dilator is capable of forced expansion of the reinforcement layer, which provides sufficient strength necessary to overcome any forces imparted by the polymeric tubing, thus controlling the cross-sectional shape of the polymeric tubing. The dilator is also capable of overcoming any forces imparted by tissues, including atrial or even ventricular myocardial tissue, through which the sheath is inserted.

In another embodiment, the sheath comprises an internal fixation device. The internal fixation device is a selectively enlargeable structure that is expanded on the exterior of the sheath portion that is resident within the stomach. In yet another embodiment, the sheath comprises an external fixation device, which is attached to the exterior of the sheath and is positioned so as to be outside the body of the patient. In a further embodiment, the sheath comprises a mechanism to controllably retract the two fixation devices toward each other so that the internal fixation device, which is inflated or expanded within the stomach, pulls the stomach wall toward the abdominal wall. The external fixation device is located external to the patient outside the skin of the abdominal incision. In another embodiment, the external fixation device is positionable at various axial locations on the sheath and is capable of being locked to the sheath at a desired location.

Another embodiment of the invention comprises a method of providing access to the stomach. The method first comprises making a percutaneous puncture incision in the abdominal wall, preferably with a hollow needle. The hollow needle, further comprising a valve at its proximal end, is advanced into the abdominal incision and into the peritoneal cavity. The needle is guided through the stomach wall and into the stomach, which is accessed from the caudal direction since it is covered by the liver when approaching from the cranial direction. A guidewire is next inserted through the fluid-static valve at the proximal end of the needle and is advanced into the stomach. The needle is removed and an expandable gastrointestinal sheath is advanced over the guidewire into the stomach. The sheath is oriented so that its distal end is positioned at the lower esophageal sphincter. The internal fixation device is expanded to prevent proximal motion relative to the stomach wall at the puncture site. The external fixation device is next expanded. The external fixation device is moved distally to close the distance between it and the internal fixation device. The stomach wall is drawn into intimate sealing contact with the interior of the abdominal wall. The dilator is next diametrically, or radially, expanded to dilate the stomach wall puncture and maximize the working lumen of the sheath. Instrumentation can now be inserted to repair the malfunctioning lower esophageal sphincter. Following completion of the procedure, the internal fixation device is collapsed or deflated and the sheath is removed from the stomach, performing whatever procedures are required to seal the stomach wall upon removing the sheath.

The expandable access sheath can be configured to bend, or flex, around sharp corners and be advanced into the stomach and be articulated so that the longitudinal axis of its distal end is parallel to the esophageal axis. Provision can optionally be made to actively orient or steer the sheath through the appropriate angles of between 20 to 120 degrees or more and to bend in one or even two planes of motion. The steering mechanism, in various embodiments, can be a curved guidewire and straight catheter, curved catheter and straight guidewire, a movable core guidewire, or a combination of the aforementioned. The expandable sheath also needs to be able to approach the target area in the gastrointestinal tract from a variety of positions. In one embodiment, radial expansion of the distal end of the access sheath from a first smaller diameter cross-section to a second larger diameter cross-section is next performed, using a balloon dilator. The balloon dilator is subsequently removed from the sheath to permit passage of instruments that may not normally have been able to be inserted into the stomach because of a small sheath size. Once the sheath is in place, the guidewire may be removed or, preferably, it may be left in place. The stomach wall is gently dilated with radial force, preferably to a diameter of 30 mm or less, rather than being axially or translationally dilated by a tapered dilator or obturator. In most embodiments, the use of the expandable gastroenterological sheath eliminates the need for multiple access system components.

In another embodiment, the expandable sheath comprises steerable members that eliminate the need for a 0.038-inch guidewire to be placed prior to sheath insertion and advancement. In yet another embodiment, a reversible fixation device, or distal anchor, is provided at the distal end of the expandable sheath. The reversible fixation device is actuated by the operator at the proximal end of the sheath. The controls at the proximal end of the sheath are operably connected to the fixation device at the distal end of the sheath by linkages, pressure lumens, electrical lines, or the like, embedded within the sheath and routed from the proximal end to the distal end. The reversible fixation device can be an inflatable structure such as a balloon, a moly-bolt expandable structure, an expandable mesh, an umbrella, or the like, preferably positioned to expand within the stomach. In an embodiment, the structure of the catheter or sheath is such that it is able to maintain a selectively rigid operating structure sufficient to provide stability against the stomach and abdominal walls to support the advancement of therapeutic instrumentation. The sheath can be selectively stiffened, at least at its distal end, to provide a non-deflecting platform for support of instrumentation, which is passed therethrough.

In another embodiment of the invention, the proximal end of the expandable sheath comprises backflow check seals or valves to prevent fluid or gas loss or retrograde flow of air into the abdominal cavity. The hub of the sheath comprises such a seal. The seal comprises an annular soft elastomeric gasket that seals against catheters, instruments, and the dilator, inserted therethrough. The seal can further comprise a valve such as a stopcock, one-way valve such as a duckbill or flap valve, or the like to prevent significant fluid or gas loss and air entry when an instrument or catheter is removed from the lumen of the expandable sheath. The soft annular seal can further comprise a mechanism to compress the inner diameter of the seal radially inward, such as the mechanisms found on Tuohy-Borst valves. The hub further comprises one or more sideport for injection of contrast media such as Omnipaque, Renografin, or other Barium-loaded solutions, for example, antacids, or the like. The dilator hub comprises a central lumen with a Tuohy-Borst valve and one or more sideports for balloon inflation, said sideports operably connected to lumens in the dilator catheter for injection or withdrawal of fluids from a balloon at the distal end of the dilator. The dilator hub, the sheath hub, or both, can also comprise a handle, lever, or trigger mechanism to enable steering mechanisms at the distal end of the dilator, the sheath, or both, respectively.

The expandable sheath, in an embodiment, comprises radiopaque markers to denote the beginning and end of the expandable region, and the middle of the expandable region. The middle of the expandable region is useful in that it can be aligned with the stomach wall during the sheath expansion procedure. The sheath can comprise radiopaque materials such as gold wire, platinum wire, tantalum wire, or coatings of the aforementioned over a malleable, stainless steel, deformable reinforcing layer. Such complete radiopaque markings are especially useful for sheath dilation insofar as they allow the operator to more clearly visualize the extent to which the sheath has been dilated once the dilator is activated. Fluoroscopic monitoring of the procedure via these radiopaque markers can be an important adjunct to direct visualization and endoscopic visualization.

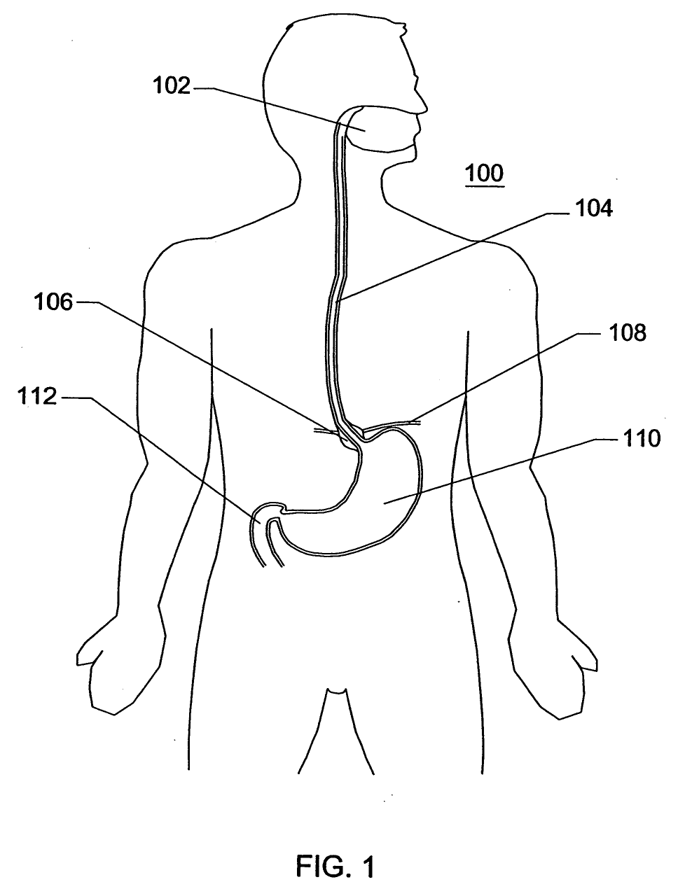

FIG. 1 is a schematic frontal (anterior) illustration (looking posteriorly) of a human patient 100 comprising a pharynx 102, an esophagus 104, a lower esophageal sphincter 106, a diaphragm 108, a stomach 110, and a descending duodenum 112. In this illustration, the left anatomical side of the body of the patient 100 is toward the right of the illustration. FIG. 1 primarily illustrates components of the upper gastrointestinal, or digestive, tract.

Referring to FIG. 1, food enters the digestive system at the mouth (not shown) and enters the pharynx 102. It then travels, by swallowing and then peristaltic motion down the esophagus 104, through the lower esophageal sphincter 104 and into the stomach 110. After leaving the stomach 110, food passes through the descending duodenum 112 on its way to the small intestine (not shown) and large intestine (not shown). The lower esophageal sphincter 106 resides just at the level of the diaphragm 108, which is the muscular wall that separates the abdominal cavity from the thoracic cavity.

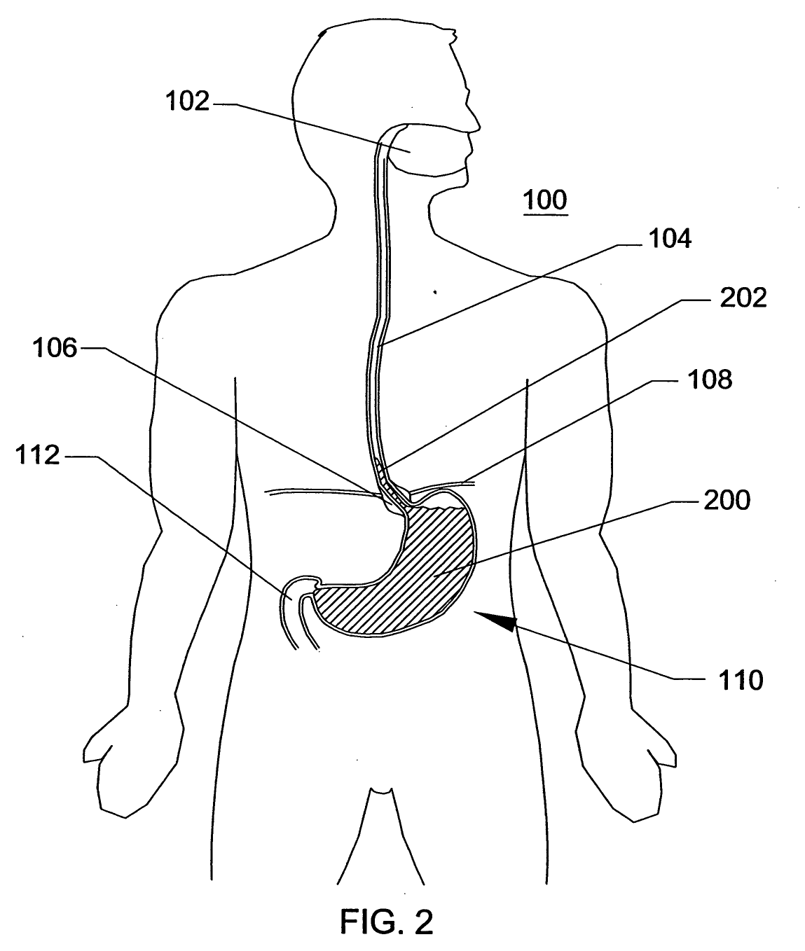

FIG. 2 is a schematic frontal illustration, looking posteriorly from the anterior side, of the patient 100 suffering from an incompetent lower esophageal sphincter 106. The gastrointestinal tract is shown with the pharynx 102, the esophagus 104, the lower esophageal sphincter 106, the diaphragm 108, the stomach 110 and the descending duodenum 112. Acidic stomach contents 200 are further shown. Regurgitated acidic material 202 or reflux of the stomach contents 200 are illustrated as residing in the lower part of the esophagus 104. While the stomach 110 is biochemically capable of handling the acidic fluids 200, the walls of the esophagus 104 are not so protected and will become damaged from repeated, or long-term, exposure to this reflux material 202.

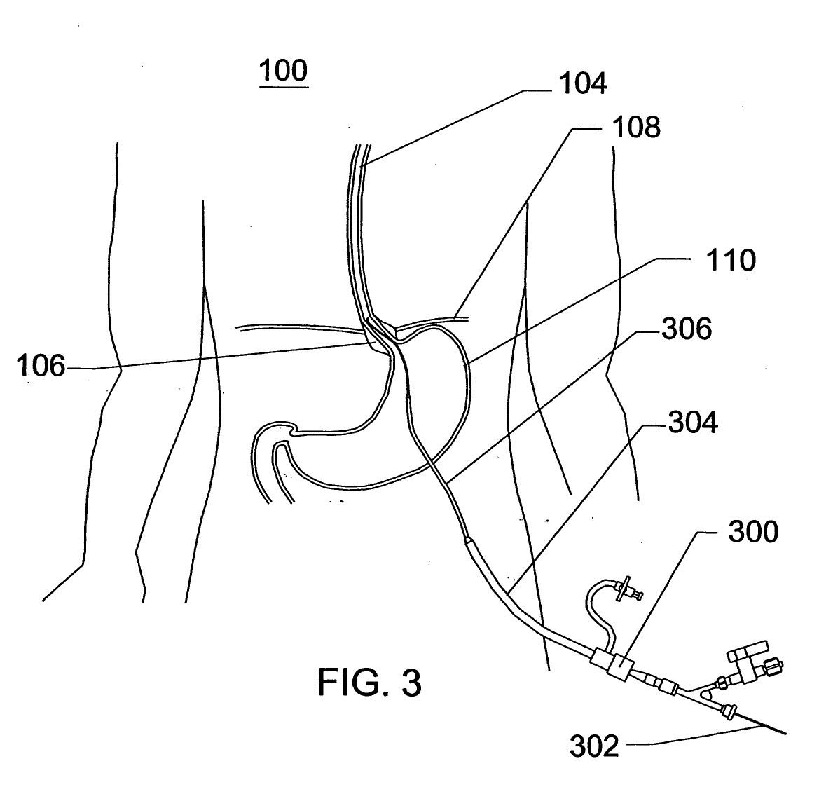

FIG. 3 is a frontal illustration, looking posteriorly from the anterior side, of the patient 100. An expandable gastrointestinal sheath 300 according to an embodiment of the present invention of has been inserted into the stomach 110 over a guidewire 302 and routed cranially toward the lower esophageal sphincter 106. The esophagus 104 and the diaphragm 108 are also shown.

Referring to FIG. 3, the guidewire 302 can be generally placed through a hollow needle, which is inserted percutaneously into the abdomen. The needle can be advanced into the stomach, through the stomach wall, after being introduced into the abdomen. Following placement of the guidewire 302, the hollow needle is removed so that catheters such as the expandable gastrointestinal sheath 300 can be advanced over the guidewire 302. The sheath 300 can also be termed an endogastric sheath.

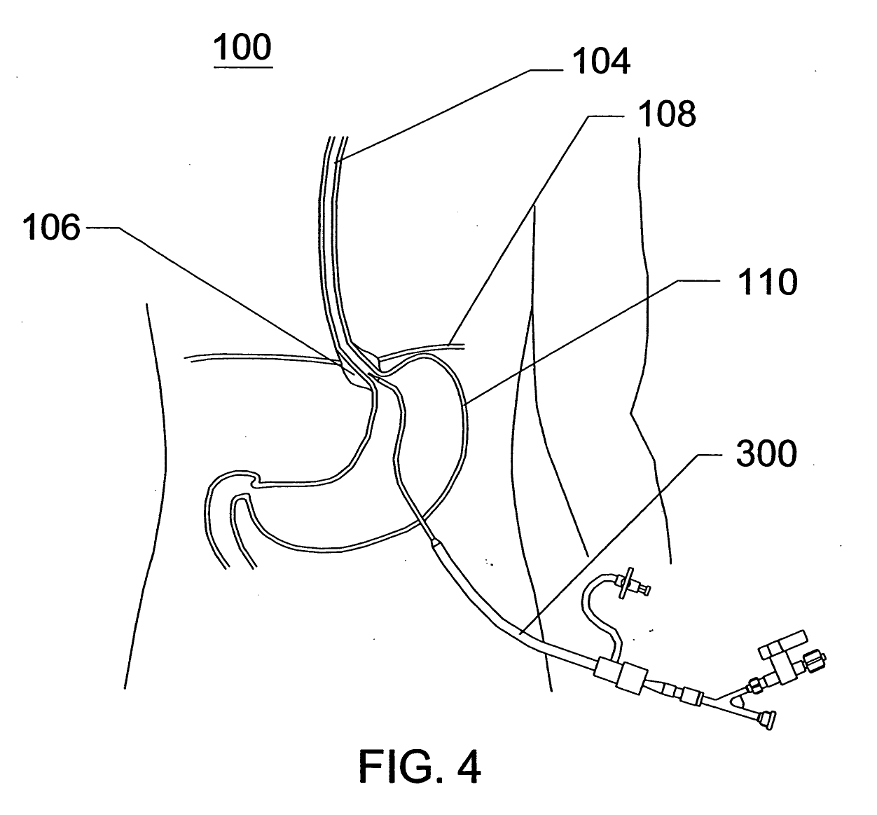

FIG. 4 is a frontal illustration, looking posteriorly from the anterior side, of the patient 100. The expandable gastrointestinal sheath 300 of has been advanced through the stomach 110 and routed cranially toward the lower esophageal sphincter 106. The esophagus 104 and the diaphragm 108 are also shown. The guidewire 302, from FIG. 3, has been removed leaving only the sheath 300. In a modified embodiment, the guidewire 302 can remain in the patient.

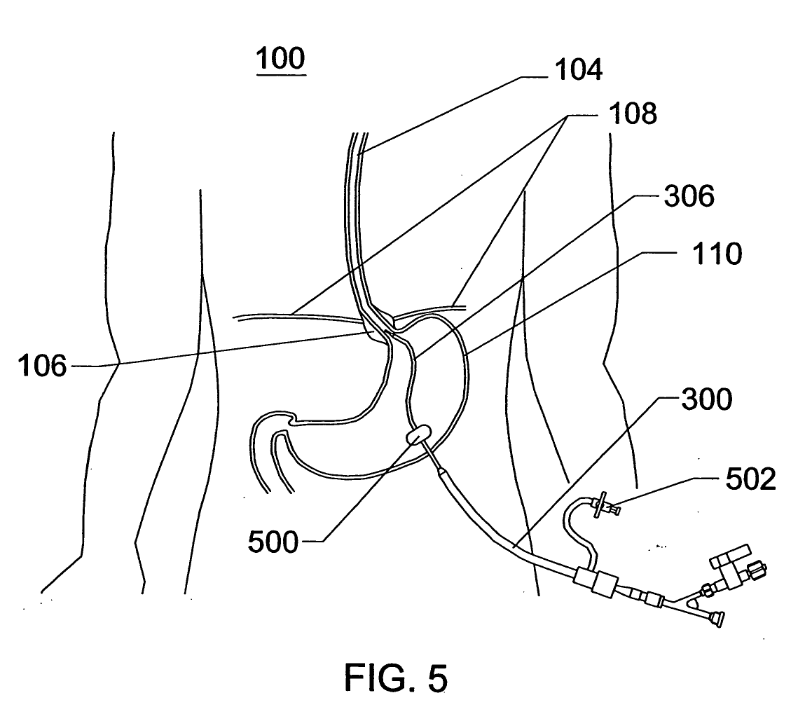

FIG. 5 is a frontal illustration, looking posteriorly from the anterior side, of the patient 100. The expandable gastrointestinal sheath 300 remains in place inside the stomach 110. The esophagus 104, the lower esophageal sphincter 106, and the diaphragm 108 are also shown. An internal fixation device 500 can be been expanded within the stomach 110 by way of pressurized fluid injection through the distal fixation balloon inflation port 502. A lumen (not shown) within, or integral to, the sheath 300 can be operably connected to the distal fixation device 500 with the inflation port 502. In this embodiment, the internal, or distal, fixation device 500 is a balloon affixed to the exterior of the sheath 300 in the expandable distal region 306. For example, the distal fixation device 500, of this embodiment, can be an elastomeric balloon similar to a Foley balloon that can be sealed to the exterior of a radially expandable part 306 of the sheath 300. The bonds, affixing the internal fixation device 500 to the sheath 300, preferably expand with the expanding sheath region 306.

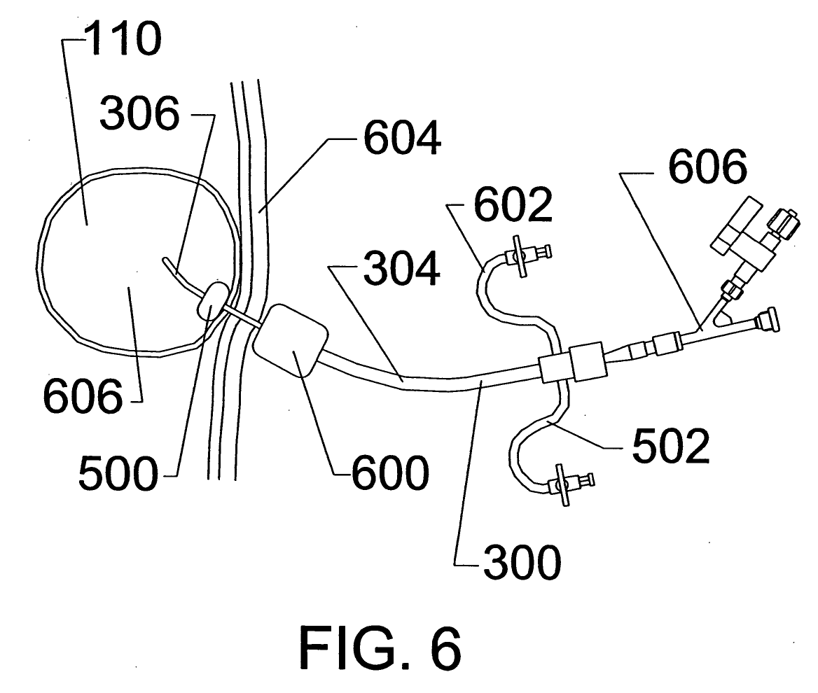

FIG. 6 illustrates a cross-sectional view of the stomach 110 and the abdominal wall 604, looking from left to right, showing the stomach lumen 606, the sheath 300, the proximal non-expandable sheath region 304, and the distal expandable sheath region 306. Also shown are the internal fixation device 500, the internal fixation device inflation port 502, an external fixation device 600, a dilator 606, and an external fixation device inflation port 602.

Referring to FIG. 6, the expandable access sheath 300 can be pre-assembled with the dilator 606. The external fixation device 600, of this embodiment, can be a balloon, (e.g., either an elastomeric balloon or a non-compliant balloon) that is affixed to the exterior of the sheath 300 in a location that is outside the abdominal wall 604. The external fixation device 600, in this embodiment, can be inflated by pressurized fluid injected into the external fixation device port 602. A separate lumen in the sheath 300 can operably connect the external fixation device inflation port 602 with the external fixation device 600. In this embodiment, the external fixation device 600 can be an everted or rolling balloon that unfurls distally as it is inflated with increased quantities of fluid. The external fixation device 600, in this embodiment, is preferably a non-compliant balloon with both the proximal and distal bond on the non-expandable region 304 of the sheath 300. The external fixation device 500 can also be a movable clamp, preferably with a gently locking collet-type lock to releasably secure the external fixation device 500 to the exterior of the proximal non-expandable region 304. In the configuration of a movable clamp, the external fixation device 500 can also comprise an extendable arm and foot to exert force against the abdominal wall 604. An particularly advantageous feature of this embodiment of the sheath 300 is the ability to retract the stomach 110 against the abdominal wall 604. Furthermore, it is beneficial to create a seal between the abdominal wall 604 and the stomach 110 so that stomach contents 200, shown in FIG. 2, cannot leak into the abdominal cavity when the stomach 110 is perforated by the sheath 300.

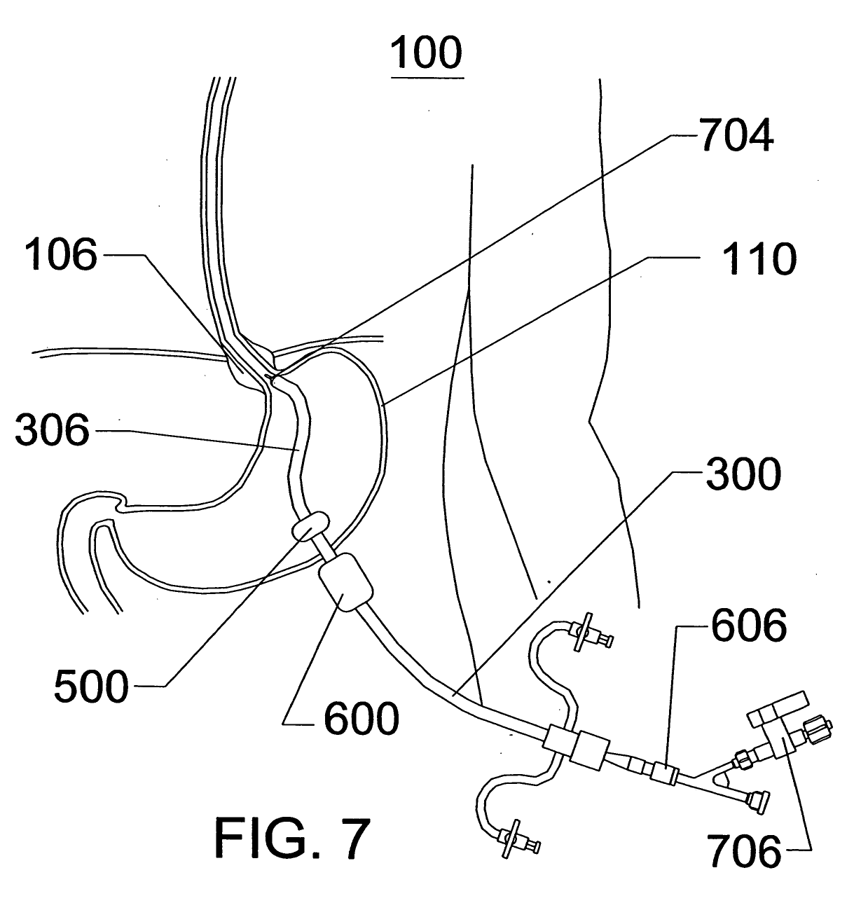

FIG. 7 is a frontal illustration, looking posteriorly from the anterior side, of the patient 100. The expandable gastrointestinal sheath 300 can remain in place inside the stomach 110. The lower esophageal sphincter 106 is also shown. In the illustrated embodiment, the expandable gastrointestinal sheath 300 further comprises the dilator 606, a dilation balloon 704, a dilation inflation port 706, an expandable distal region 306, an internal fixation device 500, and an external fixation device 600. Injection of pressurized fluid into the dilation inflation port 706 can cause inflation of the dilator balloon 704. Radial or diametric expansion of the dilation balloon 704 causes expansion of the distal expandable region 306 of the sheath 300. The stomach wall 110 can now be dilated radially following an initial puncture and then transit of the small diameter sheath 300. The use of radial dilation is considered beneficial relative to translation dilation by tapered dilators. The radial dilation allows the stomach 110 and abdominal wall 604 (FIG. 6) to be performed with relatively small expandable tips in the range of 6 to 12 French. Following transit of the stomach 110 and abdominal wall 604 through the perforation created by the hollow needle, a small sheath with a smooth, tapered, distal transition can be advanced readily through the penetration. The expandable region can then be dilated radially, opening up the abdominal wall 604 and stomach 110 penetration to any size from 12 to 30 French. Such radially dilated openings are known to heal more completely following removal of the instrument.

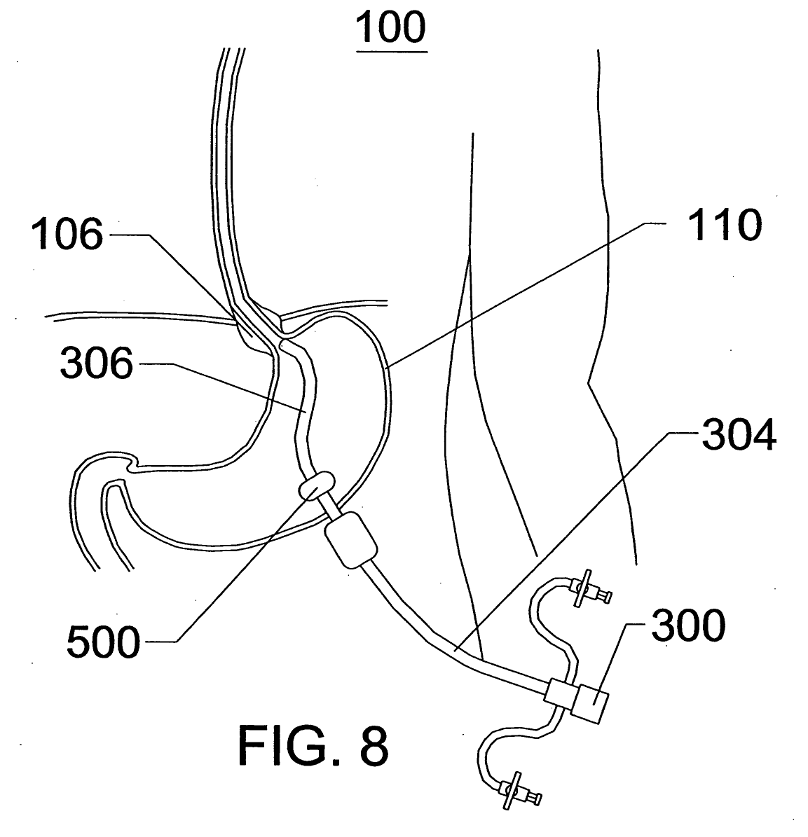

FIG. 8 is a frontal illustration, looking posteriorly from the anterior side, of the patient 100. The expandable gastrointestinal sheath 300 can remain in place inside the stomach 110. The lower esophageal sphincter 106 is also shown. The dilator 606 (FIG. 6) has been removed and withdrawn from the proximal end of the sheath 300. In this configuration, the sheath 300 retains a large, central lumen capable of passing instrumentation, catheters, or the like into the stomach 110. The size of the sheath 300 is preferably substantially the same whether in the distal expandable region 306 or the proximal non-expandable region 304. The bonds affixing the internal fixation device 500 to the distal expandable region 306 preferably have radially expanded or unfolded to continue sealing the internal fixation device of the distal expandable region 306.

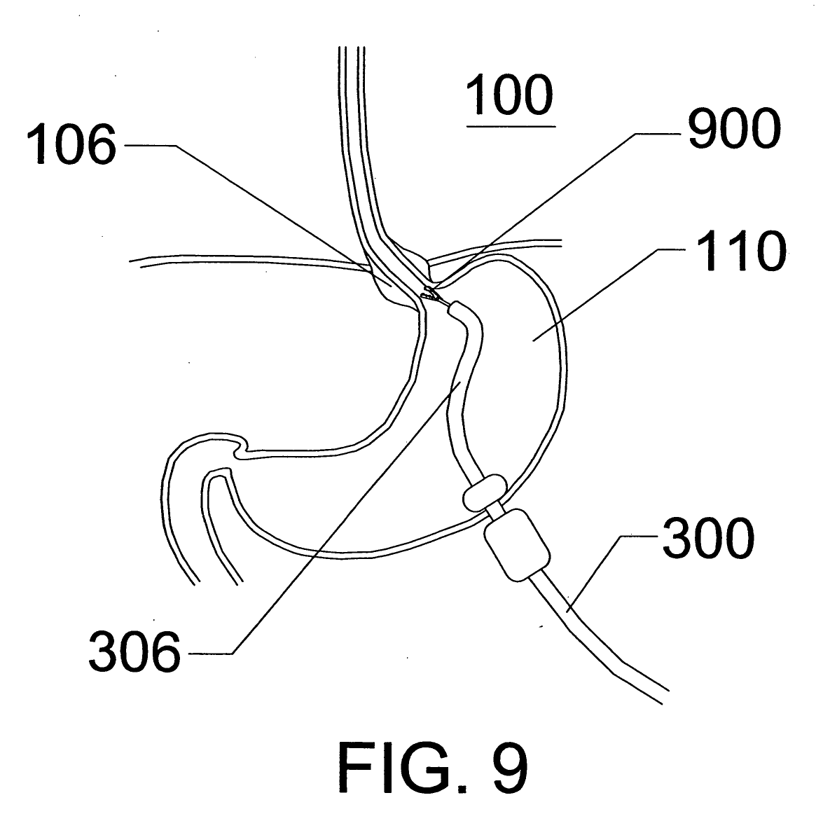

FIG. 9 a frontal illustration, looking posteriorly from the anterior side, of the patient 100. The expandable gastrointestinal sheath 300 remains in place inside the stomach 110. The lower esophageal sphincter 106 is also shown. A therapeutic catheter 900 with a grasping element can be advanced through the central lumen of the sheath 300. Because therapeutic catheters 900 can be large in diameter, they may be advantageously placed through very large sheaths such as are possible with the expandable percutaneous gastrointestinal sheath 300. In this embodiment, the therapeutic catheter 900 comprises graspers, which are being used to perform operations on the lower esophageal sphincter 106. The large sheath 300 further makes it possible for a flexible endoscope (not shown) to be advanced therethrough coincidently with the therapeutic catheter 900 so that visual monitoring of the procedure can be accomplished without the need for a second sheath system. The sheath 300 further can have a non-round, preferably pear-shaped cross-section to accommodate an endoscope and therapeutic instrument simultaneously.

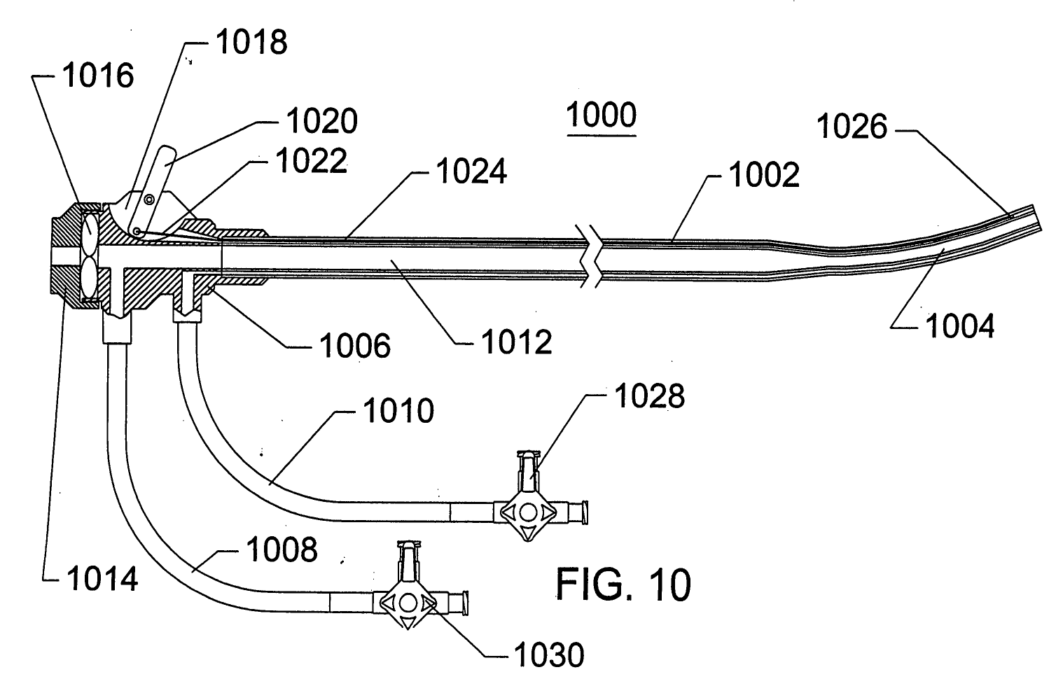

FIG. 10 illustrates a longitudinal cross-section of an articulating expandable gastric sheath 1000, which can be used in the embodiment of use described above. The articulating expandable sheath 1000 can comprise a proximal region 1002, a distal expandable region 1004, a sheath hub 1006, a central lumen 1012, a steering linkage lumen 1024, and an internal anchor inflation line 1010. The sheath 1000 can further comprise a fluid infusion line 1008, an infusion line valve 1030, an internal anchor inflation line valve 1028, a compression cap 1014, a variable valve element 1016, a lever support 1018, a steering lever 1020, a steering linkage 1022, and a steering linkage distal fixation point 1026. In this embodiment, articulation is generated by tension or a compression force in the steering linkage 1022 being applied to the fixation point 1026 affixing the steering linkage 1022 to the distal end of the distal expandable region 1004. The distal expandable region 1004 cab be flexible and can be made preferentially more flexible in the region just proximal to the distal fixation point 1026. The lever 1020 can provide mechanical advantage and can be used with ratchets, locks, friction elements, or the like to restrict movement of the lever 1020 and consequently the linkage 1022 when manual pressure is removed. The distal end of the sheath 1000 is shown bent into an arc and the lever 1020 is correspondingly moved forward to cause tension in the linkage 1022. The compression cap 1014 is threaded onto the hub 1006 and the cap controls the amount of radially inward deflection that is imparted onto the valve elements 1016 so that a seal can be created for the internal lumen 1012.

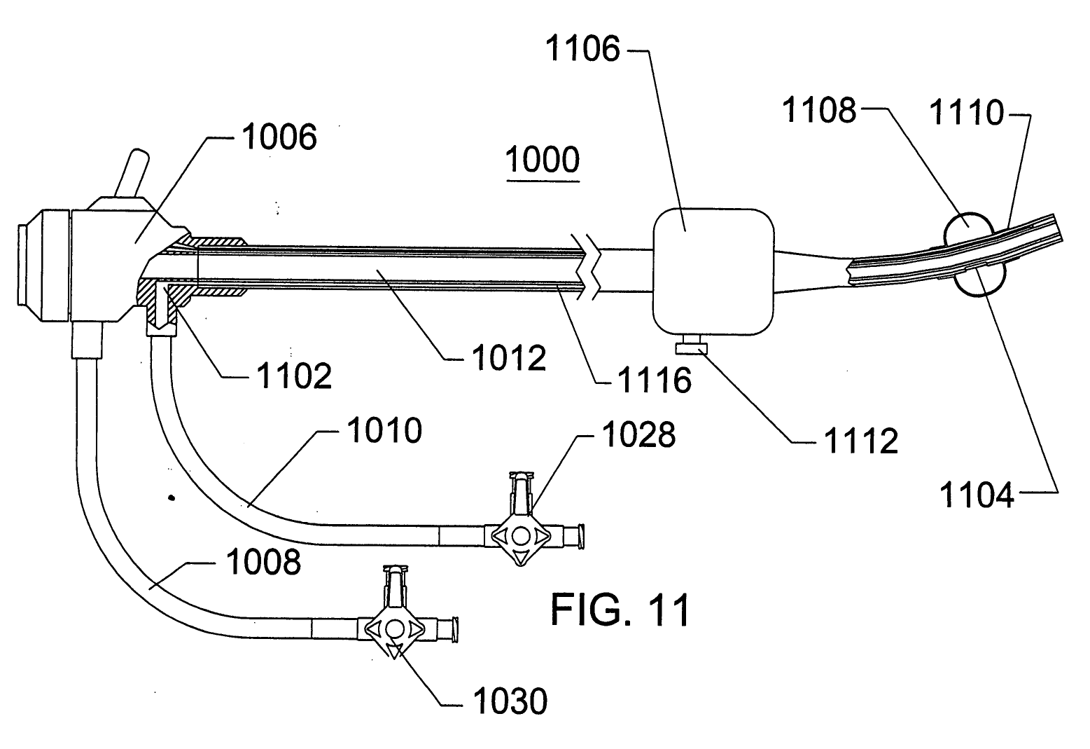

FIG. 11 illustrates a longitudinal cross-section of an embodiment of an articulating expandable sheath 1000 that further comprises a distal, internal anchor 1108, a proximal anchor 1106, and a plurality of internal anchor bonds 1110. The sheath 1000 further comprises an anchor inflation lumen 1116, a plurality of scythes 1104, an anchor inflation manifold 1102, an anchor inflation line 1010, an anchor inflation valve 1028, a hub 1006 and a central sheath lumen 1012. The distal anchor 1108 is shown as a balloon that are inflated with fluid, preferably saline, water, or radiopaque contrast media. Inflation occurs through the internal anchor inflation valve 1028, the anchor inflation line 1010, the anchor inflation manifold 1102 within the hub 1006, and the anchor inflation lumen 1116. Fluid pressure is added or removed to the balloons 1108 through the scythes 1104, which are holes or ports in the wall of the sheath 1000. The external fixation device 1106, in this embodiment, further comprises a release button 1112, which is spring-loaded and the depression of which releases an internal clamp that releaseably holds the proximal external anchor 1106 to the non-expandable region of the sheath 1000. The clamping mechanism within the external anchor 1106 can be a collet, a friction pad, a serrated grip, or the like. The external anchor 1106 needs to move longitudinally along the sheath 1000 so that it can retract the internal anchor 1108 to pull the stomach 110 against the abdominal wall.

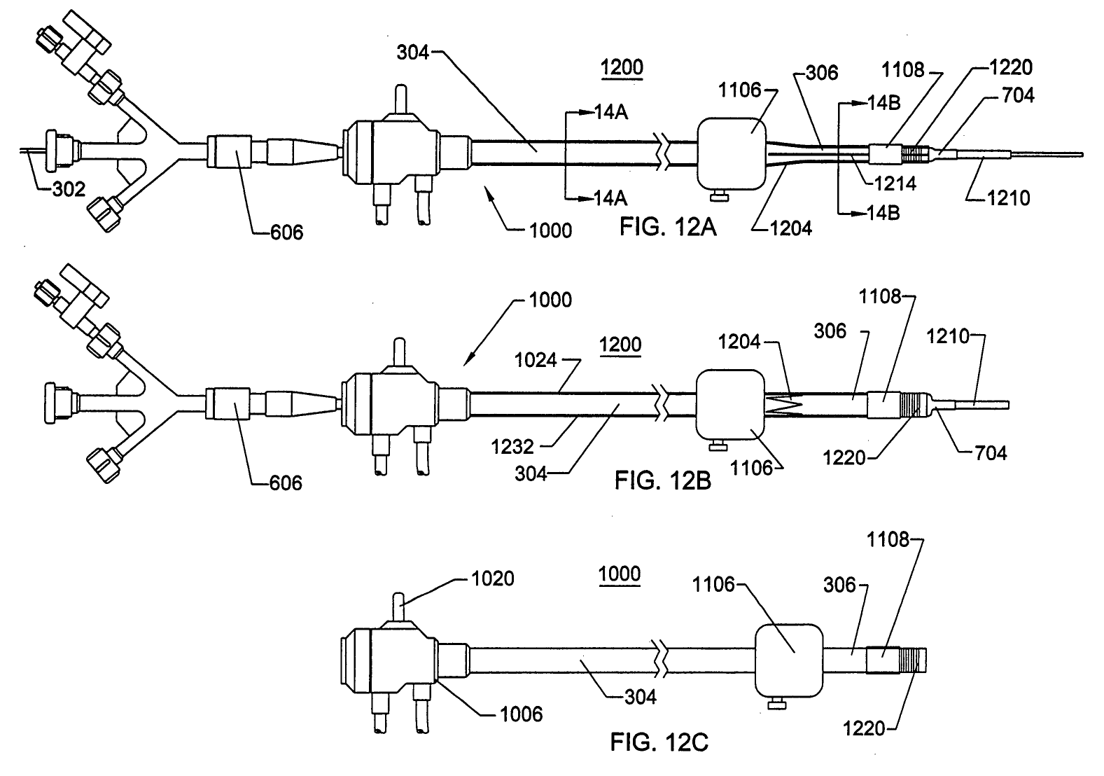

FIG. 12A illustrates a side exterior view of an embodiment of a radially expandable sheath system 1200, shown in its radially compressed configuration, comprising a dilator 606, an expandable percutaneous gastrointestinal sheath 1100, and a guidewire 302. The expandable sheath 1100 further comprises a proximal non-expandable region 304, a distal expandable region 306, an external anchor 1106, an internal distal anchor 1108, a sheath radiopaque marker 1220, a transition zone 1204, and a longitudinal fold line 1214. The dilator further comprises a dilatation balloon 704, and an inner dilation tube 1210. The external fixation device 1106 rides on, and clamps to, the proximal, non-expandable region 304 of the sheath 1000. The longitudinal fold line 1214 permits the distal expandable region 306 to be collapsed diametrically by folding its circumference. Malleable members (not shown) within the distal expandable region 306 maintain the folded configuration until it is forced to unfold at a later time. The guidewire 302 is slidably received within, and passes entirely through, a guidewire lumen (not shown) within the dilator 606.

FIG. 12B illustrates a side exterior view of the sheath system 1200 in its radially or diametrically expanded configuration. The sheath system 1200 comprises the dilator 606 and the sheath 1000. Also shown in FIG. 12B is the transition zone 1704, the proximal balloon anchor 1106, the distal balloon anchor 1108, the anchor inflation lumen 1232, the steering linkage lumen 1024, and the sheath radiopaque marker 1220. The dilatation balloon 704 is shown in its expanded, inflated configuration over the inner dilator tubing 1210. The transition zone 1204, in an embodiment, comprises a multiple chevron-shaped distal end to the non-expandable region 304 and multiple chevron-shaped distal end to the expandable region 306. The chevrons are inter-digitated and fused to form a single entity out of the two regions 304 and 306. The transition zone can also be fabricated using elastomeric or foldable materials. The steering linkage lumen 1024 and the anchor inflation lumen 1232 reside within or external to and affixed to the sheath tubing.

FIG. 12C illustrates a side exterior view of the sheath 1000 after deflation and removal of the dilator 606 (FIGS. 12A and 12B). The sheath 1000 further comprises the sheath hub 1006, the lever 1020, the proximal region 304, the distal region 306, the proximal anchor 1106, the distal anchor 1108, and the sheath radiopaque marker 1220. The transition zone 1204 is hidden underneath the external, proximal anchor 1106. The sheath 1000 is fully expanded at its distal end 306 and the distal anchor 1108 is deflated. The lever 1020 is shown in its intermediate position and the distal region 306 of the sheath 1000 is shown in its undeflected, straight configuration.

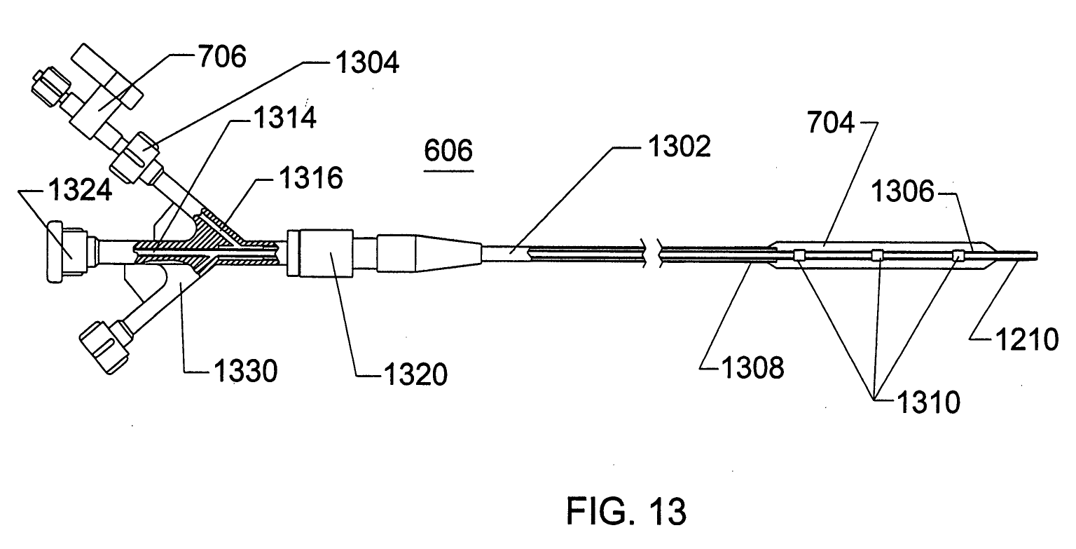

FIG. 13 illustrates a side cut-away view of a dilator 606 suitable for use with tge expandable gastric sheath 1000 (FIGS. 10 and 12). The dilator 606 further comprises a dilator hub 1320, a guidewire port with valve 1324, a guidewire lumen 1314, an inner dilator tube 1210, an outer dilator tube 1302, a dilatation balloon 704, a plurality of balloon bonds 1308, and a plurality of radiopaque markers 1310. Inflation of the dilatation balloon 704 by pressurization of the dilator inflation port 706 causes the balloon 704 to inflate. The dilator inflation port 706 is preferably a valve, such as a stopcock or one-way valve, that allows for maintenance of pressure within the balloon 704 after the pressure source is removed. The valve is connected to the hub 1320 at the hub inflation port 1304. The hub inflation port 1304 is operably connected to the hub pressure manifold 1316, which operably connects to an inflation annulus or lumen in the dilator tubing 1302. The distal opening of the inflation annulus or lumen is within the interior of the balloon 704, which is bonded 1308 to the outer catheter shaft 1302 and the inner catheter shaft 1306. The balloon 704 can be fabricated from materials such as, but not limited to, polyester, polyamide, polypropylene, irradiated polymers, polyethylene, and the like. At a 14 French diameter, the balloon 704 and the bonds 1308 can preferably hold at least 20 atmospheres (approximately 300 PSI) pressure. The balloon is pressurized by fluid under pressure such as, but not limited to, water, saline, or radiopaque contrast media. The dilator 706 can track over a guidewire up to 0.040 inches in diameter through its central guidewire lumen 1314, which extends through from the proximal end to the distal end of the dilator 706. The guidewire port and valve 1324 can be a Tuohy-Borst adapter, stopcock, duckbill valve, elastomeric ring-seal, one-way valve, or the like. The dilator hub 1320, in the illustrated embodiment, further comprises an extra injection port 1330 which could be used to inflate a second balloon or accept an instrument.

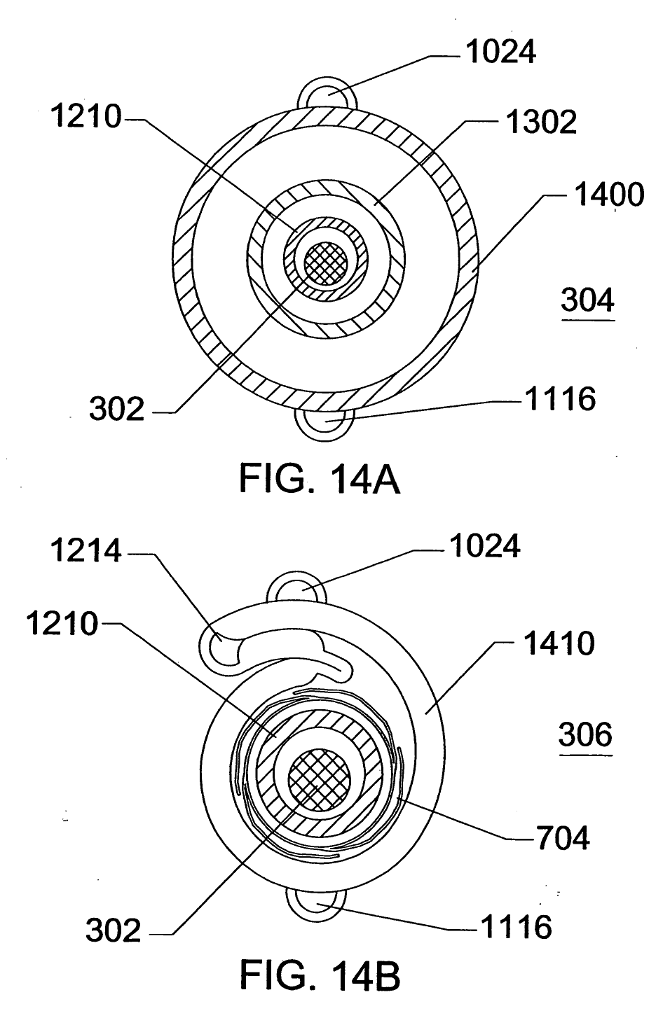

FIG. 14A illustrates a cross-sectional view of the sheath proximal end 304. The location of this cross-section is indicated in FIG. 12A. The proximal region 304 further comprises the sheath tubing 1400, the outer dilator tubing 1302, the inner dilator tubing 1210, the guidewire 302, the steering linkage lumen 1024, and the distal anchor inflation lumen 1116. The sheath tubing 1400 can be, in an embodiment, a composite tube with an inner layer of lubricious material, an outer layer, and an intermediate reinforcing layer fabricated from a coil or braid. See e.g., U.S. patent application Ser. No. 11/223,897, filed Sep. 9, 2005, entitled “Expandable Transluminal Sheath”, the entirety of which is hereby incorporated by reference herein. More than one steering linkage lumen 1024 can be used to achieve push-pull control, if separated by 180 degrees, or two axis steering if separated by 90 degrees, or 120 degrees, for example. Polymers that can be used to fabricate the composite structure of the proximal non-expandable tubing 1400 include, but are not limited to, polyethylene, polyurethane, polyvinyl chloride, polyamide, polypropylene, polyester copolymers, and the like. The reinforcing layer can be fabricated from metals such as, but not limited to, stainless steel, tantalum, titanium, nitinol, and the like. The reinforcing layer can also be fabricated from polymers such as, but not limited to, polyethylene napthalate, polyethylene terephthalate, polyamide, or the like.

FIG. 14B illustrates a cross-sectional view of the sheath 1000 (FIG. 10) distal region 306 in its collapsed configuration. The location of this cross-section is indicated in FIG. 12A. The sheath distal region 306 further comprises the distal expandable tubing 1410, the collapsed dilatation balloon 704, the anchor inflation lumen 1116, the guidewire 302, the inner dilator tube 1210, one or more longitudinal folds 1214, and the steering linkage lumen 1024. The distal expandable tubing 1410 is, in an embodiment, a composite structure with an inner layer, an outer layer, both of which are formed from polymers, and an intermediate malleable reinforcing layer, preferably fabricated from annealed metals such as, stainless steel, gold, platinum, tantalum, or the like. The metal can be coated with radiodense materials such as gold, platinum, iridium, tantalum, and the like to increase radiopacity along the entire expandable length. It is beneficial to have the entire expandable length visible under fluoroscopy so that complete expansion of the sheath 1000 can be confirmed. Polymers that can be used to fabricate the composite structure of the expandable tubing 1410 include, but are not limited to, polyethylene, polyurethane, polyvinyl chloride, polyamide, polypropylene, polyester copolymers, and the like. FIG. 14B has been magnified, relative to FIG. 14A to enhance visibility of the components so a small tube diameter in FIG. 14A will look larger in FIG. 14B.

The present invention may be embodied in other specific forms without departing from its spirit or essential characteristics. For example, the sheath may include instruments affixed integrally to the interior central lumen of the sheath, rather than being separately inserted, for performing therapeutic or diagnostic functions. The hub may comprise tie downs or configuration changes to permit attaching the hub to the abdominal skin of the patient. The dilatation means may be a balloon dilator as described in detail herein, it may rely on axial compression of a braid to expand its diameter, or it may be a translation dilator wherein an inner tube is advanced longitudinally to expand an elastomeric small diameter tube. Dilation may also occur as a result of unfurling a thin-film wrapped tube or by rotation of a series of hoops so that their alignment is at right angles to the long axis of the sheath. The embodiments described herein further are suitable for fabricating very small diameter catheters, microcatheters, or sheaths suitable for cardiovascular or neurovascular access. Various valve configurations and radiopaque marker configurations are appropriate for use in this device. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. An expandable endogastric access sheath for providing minimally invasive access to the upper digestive tract, the sheath comprising:
an axially elongate sheath with a proximal end, a distal end, and a central lumen that extends therethrough, a distal region of the sheath being located generally at the distal end of the sheath and being configured to be is expandable in circumference in response to outward pressure applied therein,
a hub coupled to the proximal end of the sheath tube;
an obturator positioned in the central lumen, the obturator configured to occlude the central lumen of the sheath during insertion, the obturator including a hub, a guidewire lumen extending through the obturator, and an expandable portion configured to expand the distal region of the sheath from a collapsed configuration to an expanded configuration;
a radially expandable distal anchor positioned on the sheath; and
a radially expandable proximal anchor positioned on the sheath proximal to the distal anchor.

2. The endogastric access sheath of claim 1 wherein the sheath comprises an outer layer, an inner layer, and a reinforcing layer wherein the outer layer and the inner layer are fabricated from polymeric materials.

3. The endogastric access sheath of claim 2 where the sheath comprises a reinforcing layer embedded within layers fabricated from polymeric materials.

4. The endogastric access sheath of claim 2 wherein the reinforcing layer is a coil of metal.

5. The endogastric access sheath of claim 2 wherein the reinforcing layer is a braid.

6. The endovascular access sheath of claim 2 wherein the inner and outer layer are fabricated from different polymers.

7. The endogastric access sheath of claim 2 wherein the length of the sheath is between 10 and 150 cm.

8. The endogastric access sheath of claim 2 wherein the inner lumen of the sheath ranges between 6 and 30 French when the distal region is fully expanded.

9. A method of proving access to a stomach of a patient, the method comprising the steps of:
inserting a guidewire through the abdominal wall into the stomach;
inserting a sheath with at least a distal region in a collapsed configuration and with a pre-inserted dilator positioned within a lumen in the sheath into the patient over the guidewire;
advancing the sheath to a treatment or diagnostic site within the stomach or upper digestive tract;
expanding the distal region of the sheath with the dilator;
collapsing the dilator;
removing the dilator from the sheath,
inserting a instrument through the lumen of the sheath into the stomach;
performing therapy or diagnosis with the instrument; and
removing the sheath from the patient.

10. The method of claim 9, wherein the step of expanding the distal region of the sheath with the dilator comprises inflating a balloon carried by the dilator.

11. The method of claim 10, wherein the step of inflating the balloon carried by the dilator comprises coupling a liquid-filled inflation device to a balloon inflation port of the dilator and infusing liquid under pressure into the dilator.

12. The method of claim 11, wherein the step of collapsing the dilator comprises withdrawing a plunger on the liquid-filled inflation device to withdraw liquid from the dilator.

13. The method of claim 9, wherein performing therapy or diagnosis with the instrument comprises performance of a Nissen fundoplication.

14. The method of claim 9, wherein performing therapy or diagnosis with the instrument comprises delivering heat or cold energy to the patient.

15. The method of claim 9, wherein the step of expanding the distal region of the sheath with the dilator comprises creating a lumen that is substantially larger than a lumen of a proximal portion of the sheath.

16. The method of claim 9 wherein the step of expanding the distal region of the sheath with the dilator comprises creating a lumen substantially smaller than that of a proximal portion of the sheath.

17. The method of claim 9, wherein the step of expanding the distal region of the sheath with the dilator comprises creating a lumen that is the same size as that of a proximal portion of the sheath.

18. The method of claim 9, further comprising expanding an internal anchor carried by the sheath within the stomach.

19. The method of claim 18, further comprising expanding an external anchor carried by the sheath outside the skin of the abdomen.

20. The method of claim 19, further comprising retracting the internal and the external anchors toward each other to seal the stomach against the abdominal wall.
